(19) United States
(12) Patent Application Publication
(10) Pub. No.: US 2005/0065599 A1
(43) Pub. Date: Mar. 24, 2005

(54) Heart valve prosthesis

(76) Inventor: Harischandra K. Mhatre, Mumbai ( Bombay) (IN)

Correspondence Address: OLSON & HIERL, LTD.;36th Floor, 20 North Wacker Drive, Chicago, IL, 60606, US

(21) Appl. No.: 10/666,456

(22) Filed: Sep. 19, 2003

(51) Int. Cl. A61F 2/24

(52) U.S. Cl. 623/2.28; 623/2.31; 264/275

(57) ABSTRACT

A prosthetic heart valve of the bi-leaflet type is provided that includes an annular structure comprised of biocompatible plastic and a pair of bearing blocks and a pair of leaflets equipped with ear-like projections that are all comprised of pyrolytic carbon. The leaflets associate pivotably with the bearing blocks, and, with the bearing blocks and the leaflets being initially held in a connected relationship, the annular structure is molded and formed about perimeter portions of the bearing blocks. The valve has a simple structure, yet is durable and reliable.

### FIELD OF THE INVENTION

This invention relates to an improved prosthetic heart valve, and more particularly to an improved bi-leaflet prosthetic heart valve made with plastic and pyrolytic carbon.

### BACKGROUND OF THE INVENTION

Bi-leaflet or double leaflet prosthetic heart valves are known to the prior art. Such a structure typically incorporates an annular member, also identified as a ring member, and two cooperating leaflets that are intended to open and close the passageway through the ring member responsive to blood flow.

Prior art bi-leaflet prosthetic heart valve structures typically suffer from various problems. One problem is that in a prior art heart valve structure commonly either the leaflets or the annular member or both must be flexed in order to assemble the valve structure. Flexing can warp, weaken or crack a flexed component of the structure, or make the flexed component susceptible to subsequent unwanted flexing in the assembled valve structure, or result in an assembled valve structure that can have a slight structural deformity owing to a tendency for a warped component not to return completely to its original unflexed configuration or condition. A heart valve structure that has been assembled by a component flexing procedure can display a tendency for the pivot ears of the leaflets to slip or even dislodge from their associated ear bearing recesses resulting in a valve that, when implanted in a patient, has leaflets which do not fully close or fully open, or do not pivot easily, or that even may seize which is a disaster for the involved patient.

Another problem is that, while it may be desirable to utilize more than one material for fabrication of various prosthetic hear valve components, such as components of pyrolytic carbon and components of plastic, the resulting assembled prosthetic valve may have poor use or structure life characteristics. The typical problem is that the different materials, owing to their formation into respective designed shapes, or their resulting assembled interrelationship relative to other components, may not function very well individually in the assembled prosthetic structure, or may function in combination to produce only an inherently weak or cumbersome prosthetic structure, so that the resulting prosthetic heart valve structure either has an undesirably short use life or functions poorly after implantation.

These and other considerations indicate the need for an improved heart valve prosthesis of the bi-leaflet type which can avoid such problems and which can incorporate pyrolytic carbon components and plastic components that are structured to work cooperatively and well together for an indefinitely long period of time and that is assembled without any flexing of components. Thereby, a durable, reliable, high quality and high performance prosthetic valve is achieved.

### SUMMARY OF THE INVENTION

The present invention relates to an improved prosthetic heart valve of the bi-leaflet type which is comprised of biocompatible components of pyrolytic carbon and plastic, which is easy and economical to fabricate and assemble, and which when assembled is durable, reliable, and performs well.

The inventive heart valve prosthesis incorporates an annular or ring-like structure comprised of a molded plastic, a pair of bearing blocks each comprised of pyrolytic carbon, and a pair of valve leaflets each comprised of pyrolytic carbon.

The annular structure incorporates two bearing block receiving windows. In each window each bearing block has an edge configuration about its perimeter which cooperatingly associates with the annular structure. Preferably, the windows and the bearing blocks have rectangular configuration.

Each bearing block has two bearing recesses defined in the flat face thereof. The two leaflets of the prosthesis each have opposed ear-like projections defined therein that are each adapted to pivotably engage a different bearing recess, though all recesses and all ear-like projections are preferably similarly sized. The bearing blocks and the windows in the annular structure are preferably rectangularly configured.

The leaflets as so engaged with the bearing blocks in the prosthesis are adapted to be disposed in and across the passageway of the annular structure. In the prosthesis, the leaflets can pivot from a valve closed position to a valve open position responsive to blood pressure applied to the passageway.

After bearing block and leaflet preparation, they are, during prosthesis fabrication, held in a desired relationship while the annular structure is molded in situ about peripheral edge portions of each bearing block. Thereby, the bearing blocks are interlocked with the annular structure with the leaflets remaining pivotable relative to the bearing blocks and the prosthetic heart valve is formed. No bending, flexing, or other distortion of components occurs.

The product prosthesis is comprised of a minimum number of components and is a relatively simple structure of great durability and reliability. The pyrolytic carbon components comprising the bearing blocks and the leaflets, and the plastic comprising the annular structure are each comprised of physiologically acceptable, non-biodegradable, implantable material.

The pyrolytic carbon components can be separately and accurately fabricated with conventional processing including use of machine tools.

Assembly of the components is simple and reliable and is accomplished without any distortion of pyrolytic carbon components.

The product prosthesis is very reliable, provides excellent service for an extended time period, and is very efficient.

The pair of pivotable leaflets employed in the prosthesis combination functions to achieve a one-way valve and to control unidirectional blood flow through the passageway of the annular member. In the valve closed position, the edge portions of each leaflets are preferably configured to abut and engage sealingly with and against adjacent surface portions contacted therewith. Each leaflet is preferably flattened and its perimeter includes an arcuately extending outside edge region, a straight inside edge region, and a pair of flattened, ear-like projections each one of which is located between a different pair of the adjacent opposite sides that extend between each end of the arcuate edge region and of the straight edge region.

Out-turned flanges at opposite ends of the annular member provide rigidity and strength. The bearing recesses achieved in each of the bearing blocks be precisely located and sized.

Small, uniform clearances between respective adjacent portions of the leaflet ears and the bearing recesses are achieved by the precise interrelationship between components, such as that between the annular structure and the associated bearing blocks. Free, smooth, self-aligning spherical bearing surfaces are achieved for pivotal hinging-type movements of the leaflets relative to the bearing blocks. End play is adjusted by selective assembly and by precise construction, as those skilled in the art will readily appreciate.

The ear-like projections of the leaflets are adapted to be held securely in the bearing recesses of the bearing blocks. A minute gap between each leaflet's ear-like projections and the bearing recesses of the bearing blocks is achieved. There is no possibility of malfunction.

The provision of pyrolytic carbon particularly in the regions of the bearing surfaces between the respective ears and the associated recesses, and the configuration of the bearing surfaces thus provided, ensures that the leaflet ears do not disengage or slip from bearings in the assembled prosthesis. The pyrolytic carbon in such regions provides a polished and hard surface.

Other and further features, purposes, objects, aims, advantages, embodiments and the like will be apparent to those skilled in the art from the present description taken with the appended drawings and the following claims.

### BRIEF DESCRIPTION OF THE DRAWINGS

In the drawings:

FIG. 1 is an isometric view of one embodiment of an annular ring structure for a prosthetic bi-leaflet heart valve of the present invention, some parts thereof being broken away and some parts thereof being shown in phantom;

FIG. 2 is a plan view of an embodiment of a prosthetic heart valve of the present invention that incorporates the annular structure of FIG. 1 and is in combination with associated bearing blocks and associated leaflets, some parts thereof being shown in section, this view being generally taken along the line II-II of FIG. 1;

FIG. 3 is an exploded isometric view of the prosthetic heart valve of FIG. 2, some parts thereof being shown in phantom;

FIG. 4 is an axial sectional view through the prosthetic heart valve of FIG. 2, this view being generally taken along the line IV-IV of FIG. 1;

FIG. 5 is a fragmentary vertical sectional view through a portion of the prosthetic heart valve of FIG. 2, this view being generally taken along the line V-V of FIG. 2 with the leaflets pivoted to a valve open orientation where the leaflets do not show in this Fig.;

FIG. 5A is a fragmentary vertical sectional view through another portion of the prosthetic heart valve of FIG. 2, this view being generally taken along the line VA-VA of FIG. 2 where the leaflets are, as shown, pivoted to a valve closed orientation;

FIG. 6 is an enlarged detailed plan view of the interior flat face of one bearing block, some parts thereof being shown in phantom;

FIG. 7 is a fragmentary transverse sectional view diagonally taken through a portion of one bearing recess of one bearing block, this view being generally taken through the region VII-VII of FIG. 6;

FIG. 8 is a longitudinal sectional view transversely taken through the region VIII-VIII of FIG. 6;

FIG. 9 is a detailed plan view of the upper, flat face of one leaflet;

FIG. 10 is a fragmentary, transverse sectional view taken through a portion of the leaflet of FIG. 9, this view being generally taken through the region X-X of FIG. 9;

FIG. 11 is a view similar to FIG. 4 but showing the leaflets in phantom in a valve open configuration;

FIG. 12 is a transverse sectional view of one embodiment of a combined mold and clamp assembly suitable for use in the in situ forming and molding of an annular ring structure for a heart valve prosthesis of the present invention, this view showing the mold and clamp assembly in association with the heart valve prosthesis after the molding therein of the annular ring structure and before the mold and clamp assembly is separated therefrom, some parts thereof being broken away;

FIG. 13 is a vertical sectional view taken along the line XIII-XIII of FIG. 12, showing the heart valve prosthesis comparably to FIG. 11, but here the leaflets are in a valve open position, and the prosthetic heart valve is in association with the combined mold and clamp assembly, some parts thereof being broken away;

FIG. 14 is a fragmentary vertical, detail, sectional view taken at an edge adjacent region of the assembly of FIG. 12, but advanced circumferentially 90 degrees relative to the longitudinal axis and the view shown in FIG. 13, showing details of the block clamping arrangement of the combined mold and clamp assembly;

FIG. 15 is a combined plan view and transverse sectional view of another embodiment of a combined mold and clamp assembly for use in the in situ molding of the annular structure, the mold and clamp assembly as shown being still holding the molded annular structure, and each of the bearing blocks as well as each of the leaflets being in a fixed association and orientation after the annular structure has been formed in the cavity of the mold and clamp assembly; some parts thereof being shown in section and some parts thereof being broken away;

FIG. 16 is a longitudinal sectional view through the assembly of FIG. 15 taken generally along the line XVI-XVI of FIG. 15; and

FIG. 17 is a longitudinal sectional view through the assembly of FIG. 15 taken generally along the line XVII-XVII of FIG. 16.

### DESCRIPTION OF THE PREFERRED EMBODIMENTS

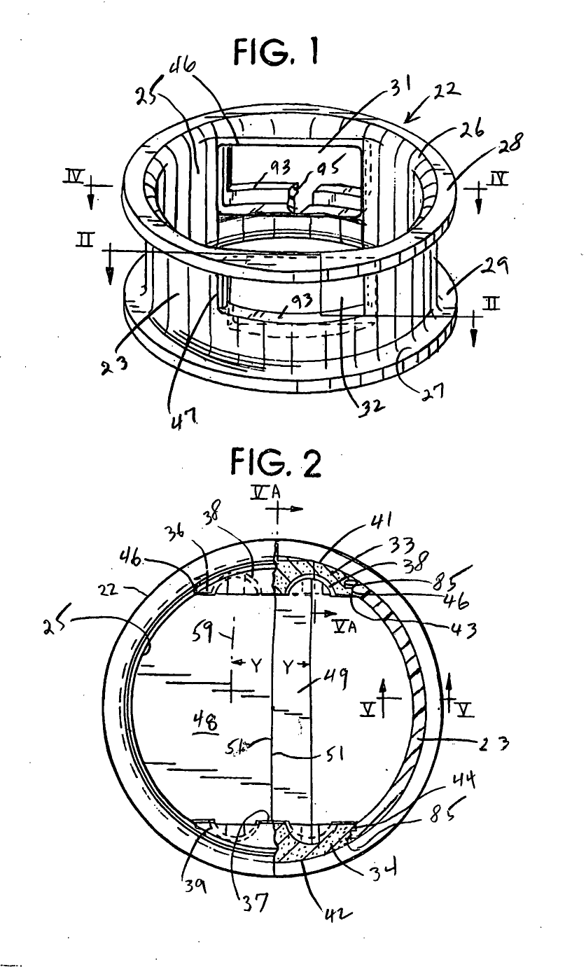

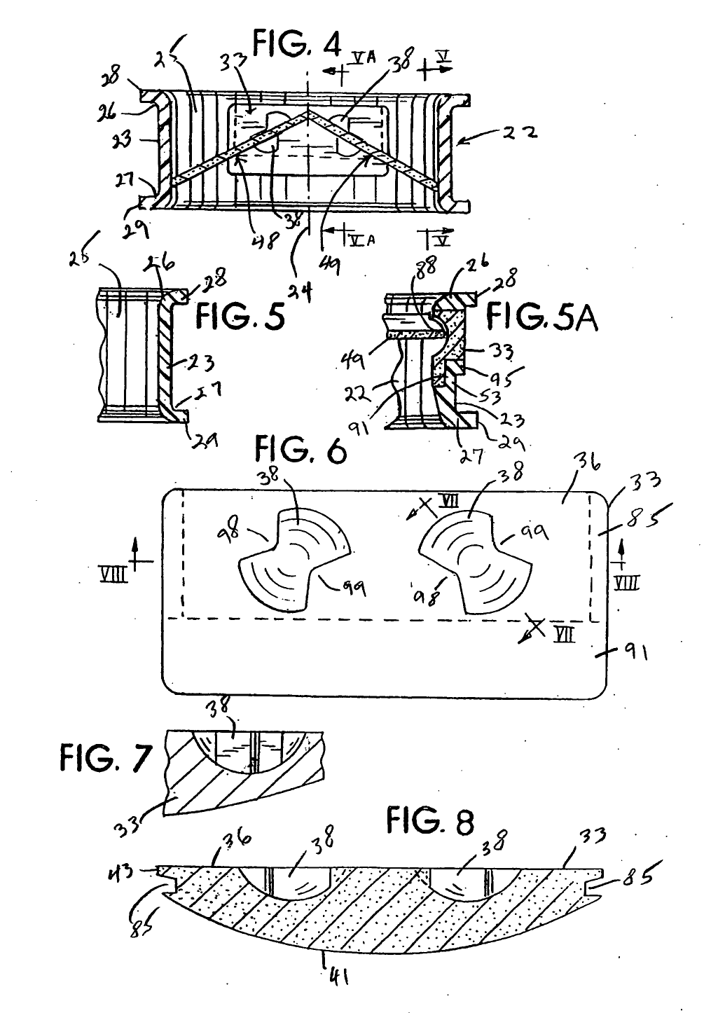

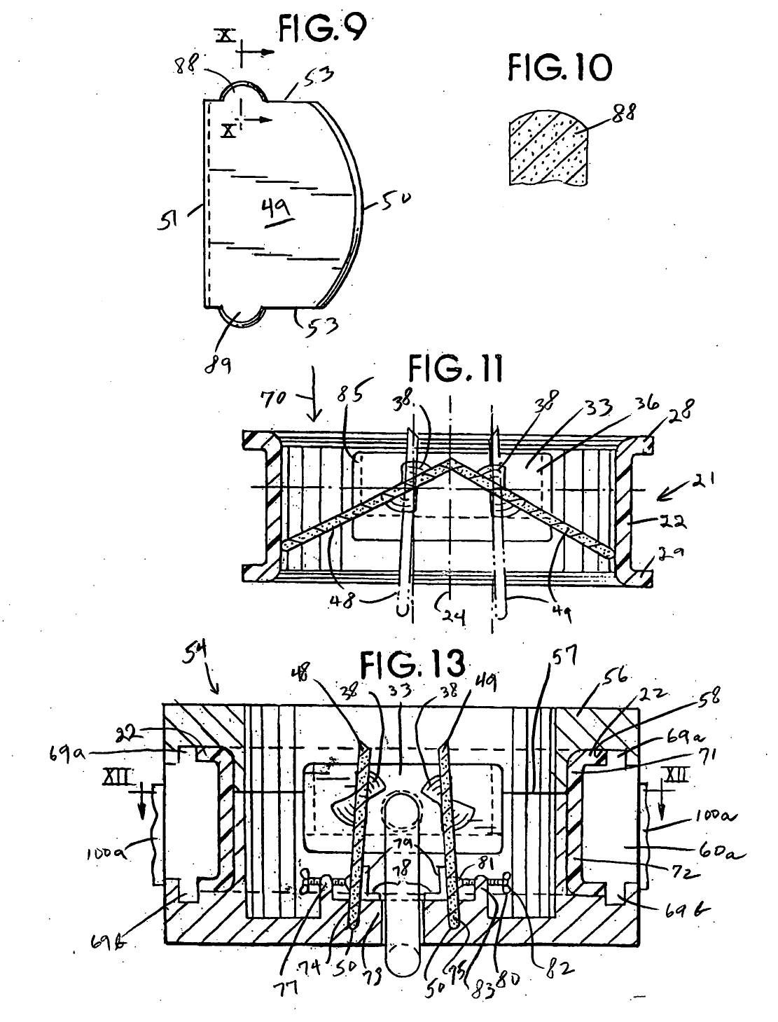

A presently preferred embodiment 21 of the inventive heart valve prosthesis is shown in FIGS. 1-14. The prosthesis 21 incorporates an annular (or ring) structure 22 which has a generally cylindrical side wall portion 23, a longitudinal axis 24 (see FIG. 4) and a longitudinal passageway 25 extending about the axis 24. Respective opposite end portions 26 and 27 of the side wall portion 23 extend circumferentially in longitudinally spaced, parallel relationship relative to each other. Each end portion 26 and 27 is provided with an outwardly and circumferentially extending integral rim flange portion 28 and 29, respectively. The rim flanges 28 and 29 extend in spaced, parallel relationship relative to each other.

The annular structure 22 has defined therein a pair of bearing block windows 31 and 32. Each window 31 and 32 is medially positioned in and extends radially through the annular structure 22 and each window 31 and 32 is located so as to be generally diametrically opposed to the other across the passageway 25.

The prosthesis 21 also incorporates a pair of bearing blocks 33 and 34. Each block 33 and 34 is adapted and configured to seat in, and engage with, a different one of the windows 31 and 32. While it is preferred that the pair of windows 31 and 32, and the pair of bearing blocks 33 and 34, each be similarly sized and configured, those skilled in the art will appreciate that different configurations can be utilized provided that the size and configuration relationship is such that one bearing block fits into one window. Each block 33 and 34 has a substantially flat interior face 36 and 37, respectively, and each face 36 and 37 has defined therein a pair of bearing recesses 38 and 39, respectively, that are circumferentially (relative to the annular structure 22) spaced from one another. All bearing recesses 38 and 39 are preferably similarly sized. The exterior face 41 and 42 of each bearing block 33 and 34 can have various configurations, but it is presently preferred to provide each of the exterior faces 41 and 42 with a curvature that generally corresponds with the curvature of the exterior surface of the sidewall portion 23.

The perimeter edge region 43 and 44 of each block 33 and 34, respectively, is configured to engage interlockingly with, and be adjacent to, the corresponding adjacent perimeter edge portions 46 and 47 of the windows 31 and 32, respectively. The manner of achieving such engagement is explained below. Preferably the edge regions 43 and 44 and the windows 31 and 32 each have a similarly sized rectangular configuration, as shown.

The prosthesis 21 further incorporates a pair of leaflets 48 and 49. In the prosthesis 21, the leaflets 48 and 49 are disposed adjacently relative to each other and transversely relative to the passageway 25. Each leaflet 48 and 49 has a generally flat body and has approximately the same size and thickness. As shown, for example, in FIGS. 2, 3 and 9, each leaflet 48 and 49 has a perimeter that includes an arcuately extending outside edge region 50, a straight inside edge region 51, and a pair of straight, transversely spaced (relative to each other) edge regions 53 that extend parallel (relative to each other). Each edge region 53 extends between and interconnects a different adjacent pair of the opposite end regions of each of the outside edge region 52 and the inside edge region 53. As shown, for example, in FIG. 9, the outside edge regions 50 are beveled to better enable the achievement of a close, abutting, preferably matching engagement thereof in the assembled prosthesis 21 with adjacent surface portions of the side wall portion 23 when the leaflets 48, 49 are each in a fully valve closed position. Similarly, the inside edge region 51 of each leaflet 48, 49 is beveled to enable the achievement of a close fitting, preferably matching, abutting engagement therebetween along a diameter of the annular structure 22 (see, for example, FIG. 2) when the leaflets 48, 49 are each in a fully valve closed position. Further, the interconnecting edge regions 53 of each leaflet 48, 49 extend perpendicularly to enable the achievement of a close fitting, minute gap or spacing, between each edge region 53 and adjacent surface portions of a bearing block 33 or 34, as the case may be, in the prosthesis 21 during pivoting movements of the leaflets 48 and 49.

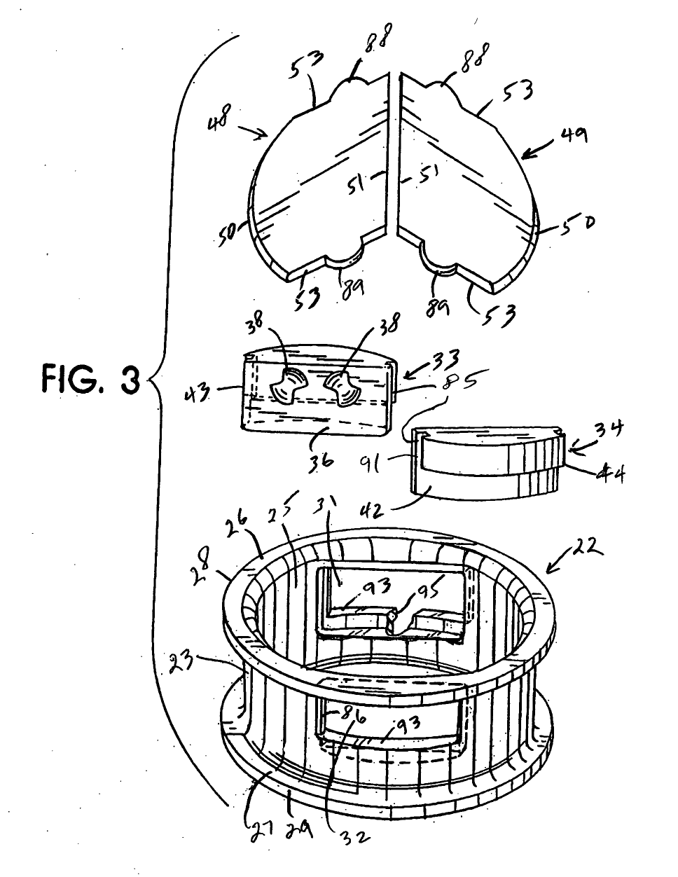

As seen, for example, in FIGS. 3 and 9, each of the leaflets 48 and 49 has a pair of outwardly extending, integral, peripherally rounded, ear-like projections 88 and 89. Each projection 88 and 89 is flattened and preferably is about as thick as the associated leaflet 48 or 49. All projections 88 and 89 are preferably similarly sized. Each ear-like projection 88 and 89 is located medially along a different one of each of the interconnecting edge regions 53. The outside perimeter curvature of each ear-like projection 88 and 89 corresponds to a spherical segment.

The interrelationship between the leaflets 48 and 49 and their respective ear-like projections 88 and 89 and the bearing blocks 33 and 34 with their respective bearing recesses 38 and 39 in the prosthesis 21 is such that each of the ear-like projections 88 and 89 is, with close spacing tolerances, receivable in, and pivotably associated with, a different one of the bearing recess pairs 38 and 39 in each of the faces 36 and 37 of the blocks 33 and 34, respectively, in the prosthesis 21. Thus, each leaflet 48, 49 is pivotable about its own pivot axis 59 (see, for example, FIG. 2) which extends between mid-regions of the ear-like projections on each of the leaflets 48, 49. The recesses 38, 39 of each flat interior face 36 of bearing block 33 and flat interior face 37 of bearing block 34 is centered along the predetermined hinging axis 59 that is in aligned relationship with the associated ear-like projection 88 or 89 in the prosthesis 21. The hinging axis 59 of each leaflet 48, 49 is parallel to a diameter of the annular structure 22 and each hinging axis 59 is equally spaced from such diameter by a distance Y (see, for example, FIG. 2) but is located on a different side thereof. In the prosthesis 21, the flat faces 36 and 37 are in spaced, parallel relationship relative to each other. When each of the leaflets 48 and 49 has its respective ear-like projections 88 and 89 associated with a cooperating respective recess 38 and 39, a small, uniform clearance preferably exists between each recess 38 and 39 and adjacent portions of the associated ear-like projection 88 and 89. Preferably, a fluid tight, pivotable joint exists between each bearing block 33 and 34 and its associated leaflets 48 and 49. Preferably the interrelationship between the bearing blocks 33, 34 and the leaflets 48, 49 in the prosthesis 21 is such that, when the leaflets 48, 49 are in their valve fully closed respective positions, a seal is achieved against the flow of blood through the annular structure 22. The edge configurations of the leaflets 48, 49 prevents leakage in the valve fully closed position, yet jamming of the leaflets 48, 49 against adjacent surfaces as a result of back pressure is prevented. Also, the relationship between bearing blocks 33, 34 is such that the leaflets 48, 49 cannot slip in the prosthesis 21.

Each recess 38 and 39 is further configured to provide a bearing surface upon which and relative to which an associated ear-like projection 88 and 89 is self-aligning. Each leaflet 48 and 49 with its ear-like projections 88 and 89 in the prosthesis 21 is allowed to swing pivotally and independently with restricted rotary oscillatory movements between a fully open and a fully closed position inclusive, and these oscillatory movements occur along the pivot axis of each leaflet 48 and 48 between its respective pair of ear-like projections 88 and 89. As those skilled in the art will readily appreciate, varying pivot excursional movements can occur during leaflet oscillations responsive to applied differential fluid (blood) pressure in a surgically implanted prosthesis 21 with the direction of blood flow being shown by illustrative arrow 70 in FIG. 11. It will be appreciated that the bearing blocks 33, 34 and the cooperatively associated leaflets 48, 49 make use of the well-established principle of “self-aligning spherical bearing.” The flattened ear-like projections 87, 89 reduce frictional losses relative to adjacent surface portions of the recesses 38, 39. Smooth pivotal movements are achieved with least effort, and with minimal loss of energy during leaflet 48, 49 oscillations. Close tolerances are achieved and are much preferred. The bell-type mouth associated with the annular structure 22 for inlet and outlet achieves smooth entry and exit for blood passage.

Each recess 38 and 39 has a region of internal surface curvature that corresponds to a spherical segment and these recesses 38 and 39 cooperate with each other and with the ear-like projections 88 and 89 for free and smooth pivotal movement of each leaflet 48 and 48 in the prosthesis 21. Each recess 38 and 39 (as illustrated, for example, in FIG. 6) has opposed side portions 98 and 99 which define side regions that act as stop means for limiting pivotal travel of the ear-like projections 88 and 89, thereby providing for the leaflets 48 and 49 desired end positions for maximum opening and closing movements. Also, inside surface portions of the annular structure 22 in the passageway 25 that are adjacent to outside regions 50 of each leaflet 48 and 49 serve as additional stop means when the leaflets 48 and 49 are in their respective fully valve closed positions. The edge regions 53 of each leaflet 48, 49 extend generally perpendicularly to the hinging axis of the associated leaflet 48 and 49. These chordal edge regions 53 function to clean blood on the adjacent flat facial regions 36 and 37 of the bearing blocks 33 and 34 during oscillations of the leaflets 48 and 49. The spherically curved edge regions of each ear-like projection 88 and 89 sweep adjacent internal spherical surfaces of each bearing recess 38 and 39. The opposed side portions 98 and 99 also serve to avoid potential stagnation of blood which might otherwise occur in what would otherwise be unused portions of the cavities of the bearing recesses 38 and 39 due to restricted pivotal movements of the leaflets 48 and 49.

The bearing blocks 33 and 34 and the leaflets 48 and 49 are comprised of pyrolytic carbon which is characteristically a hard, physiologically acceptable, non-biodegradable, implantable material. Various methods known to the prior art can be used to fabricate components comprised of pyrolytic carbon for employment in the present invention. Typically, a component with a carbon surface is heated to beyond 1,000° C. to achieve a hard and naturally polished surface. Higher temperatures give greater hardness depth relative to the surface. See, for example, Bokros U.S. Pat. Nos. 3,298,921; 3,399,969; 3,526,005; 3,547,676; and 3,676,179. For example, in one process, carbon black powder is pressed under high pressure to make bearing block and leaflet shapes. The shapes are machined and articles (components) are produced. In the present situation, the leaflets 48 and 49 and the bearing blocks 48 and 49, for example, are produced. These articles are then heated in a controlled atmosphere to 1,200° C. or above, the temperature selected being influenced by the desired structure. Thus, the resulting pyrolytic carbon components of an inventive prosthesis embodiment, such as the leaflets 48 and 49, for example, are characteristically heat treated and hardened but not coated using conventional technology. Typically, pyrolytic carbon components are inert and relatively light in weight and density.

The annular member 22 is comprised of a moldable, physiologically acceptable, non-biodegradable, implantable plastic. Various such plastics are known and can be used, as those skilled in the art will appreciate. Examples include polymethylmethacrylate and other acrylate polymers that incorporate acrylic acid or methacrylic acid; polyethylene and polypropylene including ultra high molecular weight polyethylene; polyvinylchloride (usually with stabilizers and plasticisers); polytetrafluoroethylene (PTFE or Teflon); polyesters, especially polyethylene terephthalate (PET); polyamides (including Nylon and Kelvar, especially as a reinforcing fiber in composites); polycarbonates; polyurethanes, particularly as elastomeric additives or components; certain polyaromatic semicrystalline polymers (such as “Peek Optima” and the like); silicone polymers developed for medical usage, certain ceramics, and the like.

To assemble and fabricate the prosthesis 21, the bearing blocks 33 and 34 are positioned with their respective bearing recess pairs 38 and 39 of blocks 33 and 34 engaged with the respective ear-like projection pairs 88 and 89 of the leaflets 48 and 49. The projections 88 and 89 are pivotably associated with the recesses 38 and 39. Close tolerances between adjacent surfaces are desired and preferably selected. An initial desired connected relationship between blocks 33, 34 and leaflets 48, 49 is maintained with an assembly that clamps, holds, and positions the pyrolytic carbon components (bearing blocks 33 and 34 and the leaflets 48 and 49), but that does not appreciably bend, flex, distort, or otherwise affect or mark them. With these components so held, the annular structure 22 is molded in situ by any convenient means or procedure and so is thereby formed with the edge portions 43, 44 of the bearing blocks 33, 34, respectively, being seated in the windows 31 and 32 and as now explained and illustrated. As formed, the annular structure 22 holds, positions, retains, and is in fluid tight association with, the bearing blocks 33 and 34.

The annular structure 22 is unitarily molded as a single piece component around perimeter edge portions of the bearing blocks 33, 34 with the windows 31, 32 of the annular structure 22 being formed about and defined by the perimeter edge portions 43, 44 of the bearing blocks 33, 34 respectively. The perimeter edge portions 46, 47 of each window 31, 32 are interlockingly engaged with, and effectively bonded to, the perimeter edge portions 43, 44 of the bearing blocks 33, 34, and the bearing blocks 33, 34 are held in engaged relationship with the leaflets 48, 49, respectively.

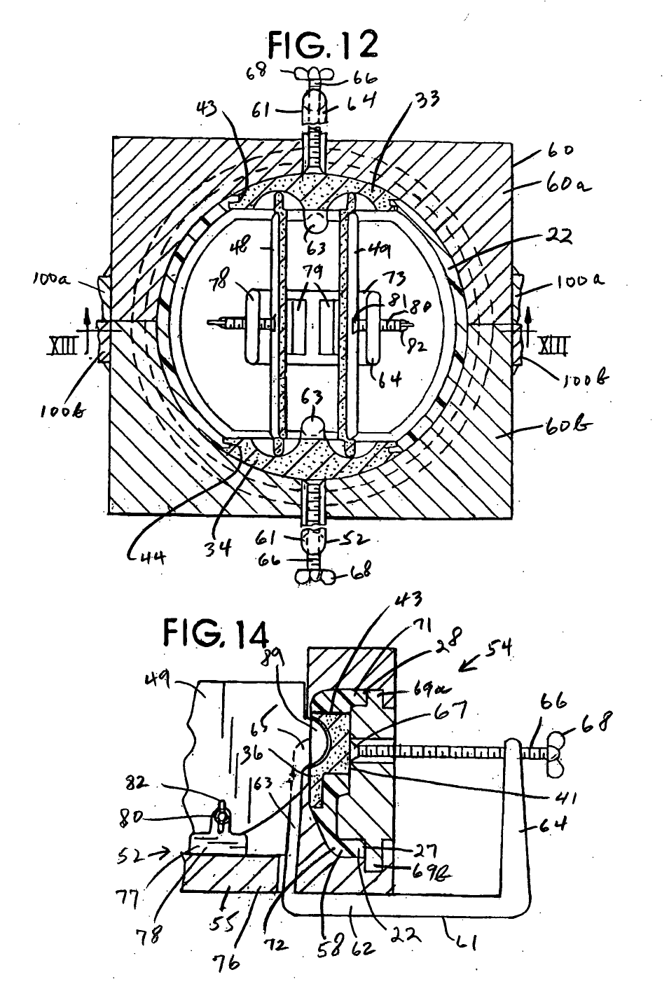

As those skilled in the art will readily appreciate, any suitable and convenient holding and molding assembly can be utilized in the practice of this invention. For present illustration and disclosure purposes, one illustrative clamping assembly 52 is shown in FIGS. 12-14. Also, as those skilled in the art will also readily appreciate, any suitable and convenient mold assembly and molding procedure can be employed. For present illustration and disclosure purposes, one illustrative mold assembly 54 is also shown in FIGS. 12-14. A present preference, as shown in FIGS. 12-14, is for the mold assembly 54 to be in combination with the clamp assembly 52.

The mold assembly 54 is here shown in a simplified form. The formation and usage of molds for plastics is well known to those skilled in the art. The mold assembly 54 includes a lower mold portion 55 which cooperatively associates with an upper mold portion 56 along a transversely (relative to the mold assembly 54) extending joint or parting line 57. Located between inner peripheral edge portions of the upper portion 56 and the lower portion 55 is a split ring structure 60 that is provided with upper and lower ridge ribs 69a and 69b that cooperatively engage receiving pockets defined in each of the inner peripheral edge portions of the upper portion 56 and the lower portion 55. The upper portion 56 and the lower portion 56 together with the split ring structure 60 define a mold cavity 58 for the annular structure 22.

The split ring structure 60 is divided into a half 60a and a half 60b. As seen, for example, in FIG. 12, a medial opposed outside edge region of each half 60a and 60b of the split ring structure 60 is fixed by welding (preferred) or the like to a different retaining arm 100a and 100b (four separate arms in all). The arms 100a and 100b are each preferably hemi-cylindrical and are adapted abut against one another lengthwise along a diameter to define a combined cylindrical configuration when abutting. When the arms 100a and 100b in the assembled mold assembly 54 are clamped together in such an abutting relationship, the split ring 60 is locked (held) in a fixed relationship as is needed to complete the definition of the cavity 58. Conventional holding and clamping means (not shown) are employed to hold in assembled combination the lower portion 55, the upper portion 56, and the split ring structure 60.

For present illustration and disclosure purposes, the cavity 58 is assumed to be completely defined and is shown holding (after being fully charged and filled with a fluid moldable plastic) a molded annular structure 22 in FIGS. 12-14. Thus, the annular structure 22 is formed in the cavity 58 of the assembled mold assembly 54 around perimeter edge portions 43 and 44 of each of the bearing blocks 33 and 34. For simplicity, the conventional location(s) are not shown in the mold assembly 54 where a moldable plastic in a fluid, usually melted, state is introduced during molding into the cavity 58 of the assembled and operable mold assembly 54 using a convenient, selected, conventional casting or injection molding procedure. Also, for simplicity, conventional means that may be employed for removing air and avoiding air pockets or heat sinks in the molded annular structure 22 molded in the mold assembly 54 are not shown.

After the molding of an annular structure 22 in the cavity 58, the clamping assembly 52 is separated from the bearing blocks 33 and 34 and the leaflets 48 and 49, and in the mold assembly 54 then the upper portion 56 and the lower portion 55 of the mold assembly 54 are separated and the split ring 60 is separated into halves 60a and 60b, thereby to enable separation and removal of the molded annular ring structure 22 from the cavity 58 together the components now associated with the annular structure 22 including the blocks 33 and 34 and the leaflets 48 and 49. The annular structure 22 as thus formed (molded) is associated and connected with the bearing blocks 33 and 34 and the leaflets 48 and 49, thereby to obtain a completed prosthesis 21.

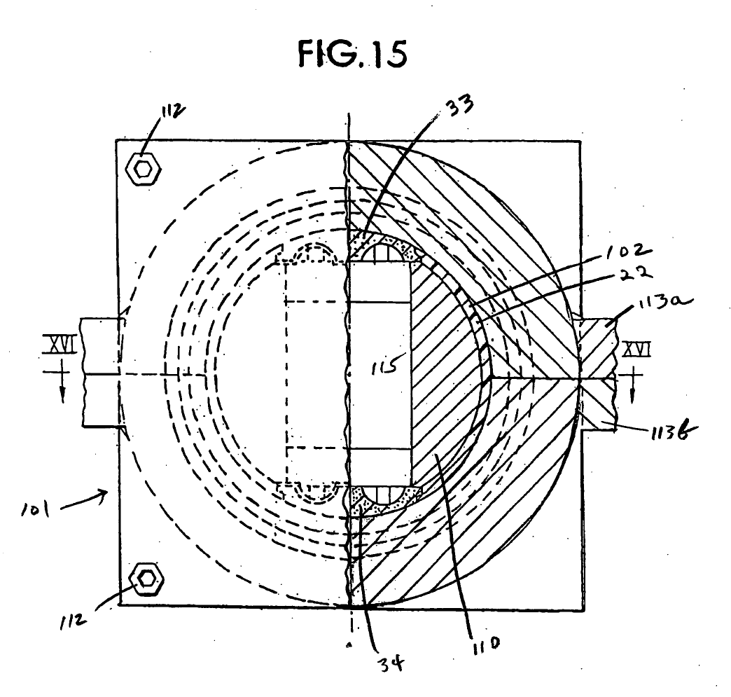

The lower portion 55 of the mold assembly 54 is associated with the clamping assembly which is generally designated as 52. The clamping assembly 52 includes a pair of C-clamp-type structures 61 (see FIGS. 13 and 14, for example). Each C-clamp structure 61 includes a base leg 62 that has arms 63 and 64 at each opposite end thereof. The arms 63 and 64 here are integrally formed with the base leg 62 and the arms 63, 64 upstand relative to base leg 62 with arms 63, 64 being in an aligned planar relationship (relative to each other). Each arm 63, 64 has a terminal outer end region. The arm 63 is provided at its outer terminal end region with a terminally generally flat faced foot 65 whose flat face is oriented so as to be generally opposed to the outer terminal end region of the arm 64. Arm 64 is engaged at its outer terminal end region with a transversely extending, threadably engaged, elongated, adjustable screw 66. Screw 66 has at its forward end a loosely journaled and affixed, self-angle-adjusting, flat faced foot 67 that, taken with screw 66, is orientable so as to be generally opposed to the outer terminal region and the flat face of foot 65 of the arm 63. At its rearward end, the screw 66 is affixed to a finger engageable, wing-like head 68 for enabling the screw 66 to be manually turned, thereby to enable adjustment of the spatial position of the foot 67 relative to and between the outer terminal end regions of the arms 64 and 63.

The base 62 of each C-clamp 61 (pair)is positioned to extend along a bottom region 76 of the lower portion 55 adjacent to an outer edge region thereof. The position of the base 62 for each C-clamp 61 is such that, when viewed in plan from along the axis 24 (which in FIG. 13 is a center point, not shown, but which is seen, for example, in the FIG. 11 view), the base 62 is generally aligned with and lies in a hypothetical, vertically oriented (relative to prosthesis 21) plain that passes generally through the axis 24 of the annular structure 22 that is being formed (molded) in the cavity 58 of the mold assembly 54. Each base 62 is preferably fastened by conventional disengageable mechanical fastening means, such as machine screws (not shown), to the bottom region 76. As shown in FIG. 14, for example, the outer (relative to lower portion 55) arm 64 projects perpendicularly upward along and in radially outwardly spaced relationship from lower portion 55. The inner (relative to lower portion 55) arm 63 projects upwardly through a hole in the bottom region 76 of the lower portion 55 and extends adjacent to an inside surface region of the lower portion 55. Preferably (as shown in, for example, FIG. 14), the inner arm 63 projects upwardly at a slight inclination angle to adapt the arm 63 to the local contour of the adjacent inside surface region of lower portion 55.

The spacing between, and the orientation of, the arms 63 and 64 is preferably such that the foot 65, the axis of the screw 66 and the foot 67 (that is associated with the screw 66) lie approximately in and along along the above indicated transverse diameter hypothetical plain. Thus, each of the bearing blocks 33, 34, as the case may be, can be positioned by a different C-clamp structure 61 between the foot 65 on arm 63 and the foot 67 on screw 66 with the flat face if foot 67 being medially adjacent to a different exterior face 41, 42 and each foot 65 being adjacent to a different interior face 36, 37. Each bearing block 33, 34 is thus locatable at, and positionable in, a window 31, 32, respectively, of the annular structure 22 defined by the cavity 58. Auxiliary mechanical positioning and measuring means (not shown) may be employed, if desired, as those skilled in the art will readily appreciate, to achieve precise positioning and spacing of the pyrolytic carbon components, within selected tolerances, preferably before these components are clamped by clamping assembly 52 components in desired positions prior to molding of the annular ring structure 22. Thus, by adjustment of the position of the foot 67 of an associated clamp 61, each of the bearing blocks 33 and 34 is positioned, held and clamped in a desired position between the outer terminal end portions of each arm 63 and 64 of a C-clamp structure 61 with the exterior face 41 and 42 adjacent the foot 67 and the interior face 36 and 36 adjacent foot 65, respectively.

The cavity 71 provided in upper portion 56 and the cavity 72 provided in lower portion 55 become abuttingly aligned in the assembled mold assembly 54 and cooperate to define windows in cavity 58 in the mold assembly 54 that correspond to each of the respective opposed bearing block windows 31 and 32 in the cylindrical side wall portion 23 of the annular structure 22. The interrelationship between each pair of windows 31, 32 thus defined by the combined cavities 71 and 72 for each bearing block 33, 34 is such that each of the bearing blocks 33, 34, as held by one of the C-clamps 61, is seated in a different one of the windows 31, 32. The respective perimeter edge regions 43, 44 of each bearing block 33, 34 forms portions of the wall surfaces defining the cavity 58 in the mold assembly 54. Thus, the relationship between each bearing block 33, 34 as held by a C-clamp structure 61, taken with the lower portion 55, the upper portion 56, and the split ring 60, is such that, when the upper portion 56 is assembled with the lower portion 55, the walls of the mold cavity 58 are fully defined except for the windows in cavity 58, as above explained.

The configuration and position of each of the perimeter edge regions 43 and 44 of the bearing blocks 33 and 34, respectively, is such that the bearing blocks 33 and 34 are held and fixed in place by the annular structure 22 after molding. Various configurations for the perimeter edge regions 43 and 44 can be employed. A present preference is for each block 33 and 34 to have a generally rectangular perimeter configuration (see, for example, FIG. 3). Preferably (as shown) there is defined in each lateral opposed short end of each edge 43, 44 of each block 33, 34 a groove 85 (see, for example, FIG. 3) which, as illustrated in, for example, FIGS. 2, 8 and 11, is sized to fit within the thickness of the cylindrical sidewall portion 23 of annular structure 22. Thus, during the molding of annular structure 22, the grooves 85 (four) each become filled with the plastic comprising the annular structure 22 to achieve a rib-like structure 86 in windows 31, 32 of sidewall portion 23 (see, for example, FIG. 3).

The windows 31 and 32 are, as shown, for example, in FIGS. 1 and 3, so that each is preferably so located in the sidewall 23 as to have an upper side thereof located adjacent to the upper flange 28 of the annular structure 22. The top side surface of the edge 43, 44 of each block 33, 34 is as shown preferably flattened and configured to extend beneath and adjacent to the contiguous lower surface portions of the upper flange 28 of the annular structure 22. Thus, the upper portion each edge 43, 44 of each window 31, 32 is defined by the plastic that comprises the annular structure 22 and comprises a relatively broad expanse that radially extends (relative to the annular structure 22) from the upper edge of the flat interior face 36, 37 of each block 33, 34 to the contiguous adjacent outer arcuate edge portion of the upper flange 28 of the annular structure 22. Such broad expanse, in addition to improving the fixed association desired between each block 33, 34 and the annular structure 22, is believed to strengthen the association and also to provide each block 33, 34 with the capacity to resist possible tilting movement of blocks 33, 34 relative to the axis 24 that might otherwise weaken or even dislodge the desired fixed association between the annular structure 22 and each block 33, 34. Optionally, the flattened top surface of each block 33, 34 can be roughened or otherwise shaped, if desired (not shown), to achieve a desired surface for formation of an annular structure 22.

The back exterior face 41, 42 and adjacent surface portions of the perimeter edge 43, 44 of each block 33, 34 is as shown (see for example FIG. 3) preferably formed with an outwardly projecting flange portion 91 and whose outside face extends arcuately and parallel to the adjacent face 41, 42. Owing to the cooperative association between the respective blocks 33, 34 and the lower portion 55 of mold assembly 54, the cavity 58 is configured so that, along the bottom edge of the perimeter 46, 47 of each window 31, 32, a mating upstanding shoulder 93 is provided which has a terminal outwardly extending flange 95 (relative to each window 31, 32, as shown in FIGS. 5A and 14, for example). Thus, when the annular structure 22 is molded in the cavity 58, the annular structure 22 includes this shoulder 93 and flange 95 at the backside edge region of each block 33, 34. This interrelationship between the bottom edge and adjacent backside regions of each block 33, 34 and each window 31, 32 in annular structure 22 is believed to provide a desirable locking inter-engagement between adjacent respective portions of each block 33, 34 and the annular structure 22.

Other arrangements and configurations for the edge surfaces 43, 44 of the blocks 33, 34 and of the windows 31, 32, respectively, can be employed, if desired.

As part of the clamping assembly 52, the upper surface of the central bottom region of the lower portion 55 of mold assembly 54 is provided with a raised (vertically thickened) platform region 73. A pair of diametrically (relative to the annular structure 22) spaced channels 74, 75 is defined in platform 73 so that each channel 74, 75 extends straight lengthwise, transversely relative to a hypothetical cord structure extending across portions of the annular structure 22, and downwardly into platform 73 from the upper central surface thereof so that, with increasing channel depths, the channel 74 slightly diverges from, and is somewhat inclined relative to, the channel 75 (see, for example, FIG. 12). Cross-sectionally, each channel 74, 75 extends in a generally spaced, parallel relationship relative to the other, and each channel 74, 75 is generally equally spaced from the longitudinal axis 24 (see FIG. 11) of the annular structure 22 as defined by the cavity 58. Each channel 74, 75 is adapted to receive therein a medial region along an edge 50 of each leaflet 48, 49 with the ear like projections 88, 89 of each leaflet 48, 49 being concurrently positioned for pivotal movement in bearing recesses 38, 39 of each bearing block 33, 34 (as such is held by a C-clamp 61 as above described; see, for example, FIG. 14) To hold each of the leaflets 48, 48 in a fixed position so as to achieve a desired close tolerance between the adjacent respective surface portions of the bearing blocks 33, 34 and the bearing recesses 38, 39, the leaflets 48, 49, as associated with the projections 88, 89, are clamped in a desired position by respective ones of a pair of relatively small C-clamp assemblies 77 that are each associated with the platform 73.

Each C-clamp assembly 77 includes a foot member 78 that is mounted on the platform 73 so as to be adjacent to, but equally spaced from, the longitudinal axis 24 of annular structure 22 as defined by the cavity 58. Each foot member 78 is parallel to the other. Each foot member 78 is also adjacent to a different channel 74, 75 (see FIG. 12). Each foot member 78 has an upstanding leg 79, each leg 79 being slightly inclined to conform to the slope established by the adjacent channel 74, 75. Along opposed outside edge portions of the platform region 73 a post 83 upstands. Through an upper end region of each post 83 an elongated screw 80 threadably extends generally horizontally.

Each screw 80 has its forward end loosely journaled and affixed to a self-angle-adjusting, flat faced foot 81 that, taken with screw 80, is oriented so as to be generally opposed to an upper end region of the each leg 79. At its rearward end, the screw 80 is affixed to a finger engageable, wing-like head 82 for enabling the screw 80 to be manually turned, thereby to adjust the spatial position of the foot 81 relative to and between the outer terminal end regions of the leg 79 and the post 83.

The spacing between, and the orientation of, the terminal end regions of the leg 79 and the bracket 85 is such that each one of the leaflets 48 and 49 can be positioned, oriented and held by a different leg 79 and an adjacent foot 81, the foot 81 being adjustable by screw 80. Thus, by adjustment of the position of the foot 81, each of the leaflets 48, 49, with their respective projections 88, 89 engaged with the bearing recesses 38, 39, can be positioned and held in a desired position between the bearing blocks 33, 34, as desired, by a different C-clamp assembly 77.

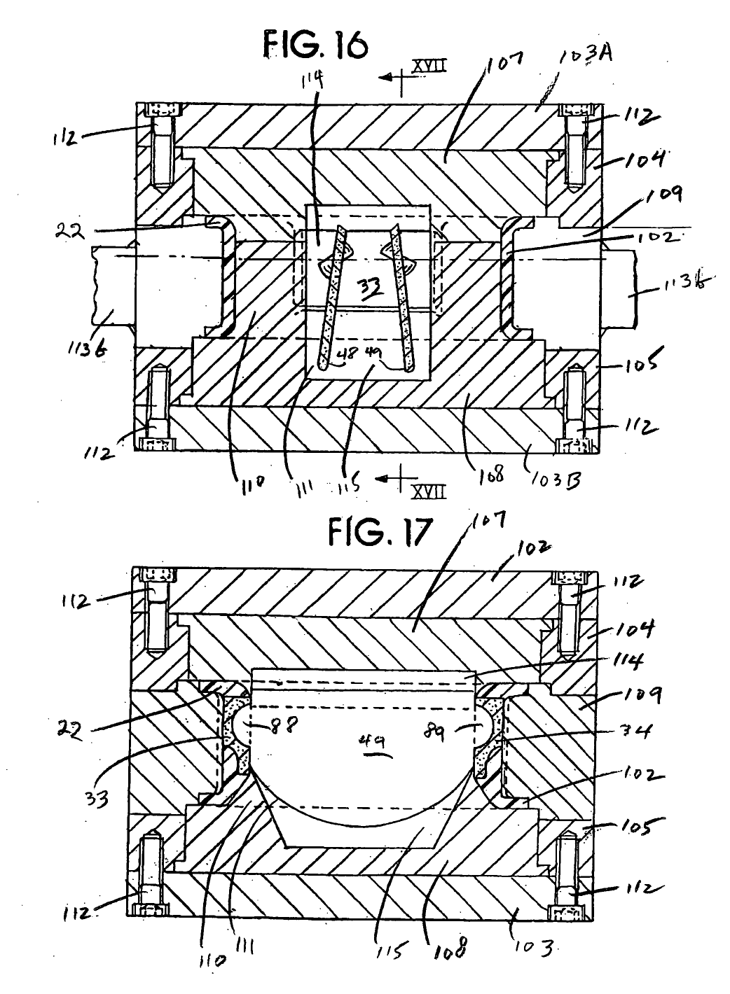

Another suitable holding and molding assembly embodiment 101 is illustrated in FIGS. 15-17. In assembly 101, the components coact to provide both the cavity 102 in which the plastic annular structure 22 is molded and the holding, retaining and positioning function for the pyrolytic carbon components blocks 33, 34 and leaflets 48, 49.

The assembly 101 incorporates upper and lower cap plates 103A and 103B each with interior peripheral surface portions that abuttingly engage an adjacent side edge region of each of an upper and lower forming ring 104 and 105, respectively. The medial region of each ring 104 and 105 is fitted with an upper and lower circular forming plate 107 and 108. The middle peripheral region of the assembly 101 is provided with a split ring 109 that defines outside wall portions of the cavity 102 and that is located radially in, and fitted between, the upper and lower forming rings 104 and 105. The lower forming plate 108 includes an integral, upstanding, central, plateau-like region 110 which is provided with a relatively large central aperture 111 that longitudinally extends therethrough and downwardly into forming plate 108. In combination with lower portions of the forming plate 107, the outside circumferential wall portions of the region 110 define inside wall portions of the cavity 102. The split ring 109 is comprises of half 109a and half 109b. A medial, outside, opposed region of each half 109a and 109b is attached, preferably by welding, to a clamping arm 113a and 113b (four separate arms in all) which are comparable to the arms 100a and 100b (above) and which function similarly so that the split ring can be held in a closed position as needed to define the cavity 102 yet permit separation of a molded annular structure 22 from the apparatus 101, as those skilled in the art will readily appreciate. To retain the components in association, allen bolts 112, preferably four, extend vertically through each plate 102 and 103 into threaded engagement with the adjacent ring 104 and 105, respectively.

Inside facial portions of the ring 109 and outside facial portions of the region 110 cooperate to define at opposed locations the bearing block windows 31 and 32 provided in the cylindrical side wall portion 23 of the annular structure 22 that is defined by the cavity 102. Each bearing block 33 and 34 is positioned in a window 31 and 32, respectively. Edge portions of each of the ring 109 and the region 110 that are adjacent to the apertures defined by ring 109 and region 110 that provide each window 31 and 32 are sized so as to slightly overlie edge portions of each of the bearing blocks 33 and 34. Thus, in the assembled assembly 101, each bearing block 33 and 34 is held in position at each window 31 and 32 defined by the ring 109 and region 110.

Prior to positioning of the bearing blocks 33 and 34 in the assembly 101, the leaflets 48 and 49 are associated with the bearing blocks 33 and 34 with each projection 88 and 89 being located in a different bearing recess 38 and 39 as above explained. The lower surface region of the forming plate 107 and the upper central surface region of the lower forming plate 108 are each provided with a cavity 114 and 115, respectively, as shown. Thus, when the assembly 101 is assembled with the bearing blocks 33 and 34, and edge portions of the bearing blocks 33 and 34 are held by portions of the region 110 and the ring 109, as above explained, and the leaflets 48 and 49 are associated with the bearing blocks 33 and 34, the leaflets 48 and 49 extend unimpeded but inclined through the aperture 111 and into the respective cavities 114 and 115 defined in the forming plates 107 and 108. The annular structure 22 can then be molded after which a completed prosthesis 21 is separated from the assembly 101 by disassembly of assembly 101.

The assembly 101 is here shown in a simplified form. The formation and usage of molds for plastics is well known to those skilled in the art. The assembly 101, if desired, like the mold assembly 54, may include additional portions and features. For present illustration and disclosure purposes, the cavity 102 is assumed to be completely defined by the assembly 101 and is shown holding (after being fully charged and filled with a fluid moldable plastic) a molded annular structure 22. Thus, the annular structure 22 is formed in the cavity 102 of the assembly 101 around perimeter edge portions 43 and 44 of each of the bearing blocks 33 and 34. For simplicity, the conventional location(s) are not shown in the assembly 101 where a moldable plastic in a fluid, usually melted, state is introduced during molding into the cavity 102 of the assembly 101 using a convenient, selected, conventional casting or injection molding procedure. Also, for simplicity, conventional means that may be employed for removing air and avoiding air pockets or heat sinks in the molded annular structure 22 molded in the mold assembly 54 are not shown.

The assembly 101 illustrates an alternative arrangement for the annular structure 22 in the vicinity of the bearing blocks 33, 34. After molding of an annular structure 22 in an assembly 101, the assembly is disassembled by removing the alien bolts 112 and separating the split ring 109, as those skilled in the art will appreciate.

If desired, for example, the bottom outside region of each block 33, 34 may be provided with a different configuration from that shown, for example, in FIGS. 3, 5A, 14 and 17 and also the radial thickness of the sidewall portion 23 of the annular structure 22 may be provided with a different thickness. Because the inside surface portions of the sidewall 23 are cylindrical while the respective interior faces 36, 37 of the blocks 33, 34 are flat, as described above, the configuration of the sidewall 23 in the vicinity of the blocks 33, 34 can be adjusted so as to have, for example, a maximum thickness that corresponds to the medial thickness of a bearing block 33, 34 or a minimum thickness that corresponds to the average radial thickness of the sidewall 23. In the latter situation, the mid-region of each bearing block 33, 34 projects radially into the passageway 25 as those skilled in the art will readily appreciate. Various relationships between the bearing blocks 33, 34 and the sidewall 23 can be utilized without departing from the spirit and scope of the invention.

Various other and further embodiment applications, structures and the like will be apparent to those skilled in the art from the teachings herein provided and no undue limitations are to be drawn therefrom.

1. A prosthetic heart valve assembly comprising in combination:
(a) an annular structure comprised of a moldable, physiologically acceptable, non-biodegradable, implantable plastic and having a longitudinal cylindrical passageway defining a sidewall portion that has a pair of bearing block receiving windows defined therein, each said window being generally diametrically opposed relative to the other;
(b) a pair of bearing blocks, each said bearing block being comprised of pyrolytic carbon and being seated in a different one of said windows, and each said bearing block having a substantially flat interior face, a pair of circumferentially spaced bearing recesses defined in said interior face, and perimeter edge portions interlockingly and sealingly engaged with said so seated window; and
(c) a pair of leaflets, each said leaflet being comprised of pyrolytic carbon and being adjacent to the other thereof and transversely disposed across said passageway, each said leaflet having a generally flat body portion whose perimeter includes a generally arcuately extending outside edge region, a straight inside edge region, and a pair of straight, spaced, parallel, side edge regions that each extend between and join a different adjacent terminal portion of each of said outside and said inside edge regions, and each one of said side edge regions having a medially located, integral, outwardly extending, flattened, peripherally rounded, ear-like projection;
the interrelationship between said leaflets, said ear-like projections and said bearing blocks and said bearing recesses being such that each of said ear like projections is receivable in and pivotably associated with a different one of said bearing recesses in each of said bearing blocks whereby said leaflets can pivot between valve open and valve closed positions; and
said annular structure is unitarily molded in situ about said perimeter edge portions.

2. The prosthetic heart valve assembly of claim 1 wherein said annular structure has a generally cylindrical sidewall portion that defines said longitudinal passageway and that has respective opposite end portions that extend circumferentially in longitudinally spaced, parallel relationship.

3. The prosthetic heart valve assembly of claim 2 wherein said respective opposite end portions each have an outwardly and circumferentially extending rim flange portion.

4. The prosthetic heart valve assembly of claim 1 wherein said bearing blocks and said leaflets are initially held in a connected relationship wherein said interior face of each said bearing block is in spaced, parallel relationship relative to the other thereof, and each said ear-like projection is pivotably associated with a different bearing recess.

5. The prosthetic heart valve assembly of claim 1 wherein
(a) each said leaflet is responsive to fluid pressure applied against an upstream side of said passageway, and
(b) said leaflets cooperate so that, responsive to changes in said applied fluid pressure, each pivots between a first position where said passageway is substantially fully closed to a second position where said passageway is substantially fully open, thereby defining respective valve closed and valve open configurations relative to said passageway.

6. A prosthetic heart valve assembly comprising in combination:
(a) an annular structure comprised of a moldable, physiologically acceptable, non-biodegradable, implantable plastic and having sidewall portions that define a generally cylindrical, longitudinal passageway therethrough, opposite end portions that extend circumferentially in longitudinally spaced, parallel relationship, and circumferentially extending rim flange portions defined about each said opposite end portion, and having a pair of bearing block receiving windows defined in said sidewall portions, each said window being generally diametrically opposed relative to the other;
(b) a pair of bearing blocks, each said bearing block being comprised of pyrolytic carbon and being seated in a different one of said windows, and each said bearing block having a substantially flat interior face, a pair of circumferentially spaced bearing recesses defined in said interior face, and perimeter edge portions interlockingly and sealingly engaged with said respective seated window; and
(c) a pair of leaflets, each said leaflet being comprised of pyrolytic carbon and being adjacent to the other thereof and transversely disposed across said passageway, each said leaflet having a generally flat body portion whose perimeter includes a generally arcuately extending outside edge region, a straight inside edge region, and a pair of straight, spaced, parallel, side edge regions that each extend between and join a different adjacent terminal portion of each of said outside and said inside edge regions, and each one of said side edge regions having a medially located, integral, outwardly extending, flattened, peripherally rounded, ear-like projection, said ear-like projections of each said leaflet being pivotably engagable with a different one bearing recess of each one of said bearing blocks;
the interrelationship between said leaflets, said ear-like projections and said bearing blocks and said bearing recesses being such that each of said ear like projections is receivable in and pivotably associated with a different one of said bearing recesses in each of said bearing blocks whereby said leaflets can pivot between valve open and valve closed positions; and
said annular structure is unitarily molded in situ about said perimeter edge portions.

7. The prosthetic heart valve assembly of claim 6 wherein said bearing blocks and said leaflets, during said in situ molding of said annular structure, are initially held in a connected relationship with said interior face of each said bearing block is in spaced, parallel relationship relative to the other thereof, and with each said ear-like projection being pivotably associated with a different bearing recess in each said bearing block.

8. The prosthetic heart valve assembly of claim 6 wherein
(a) each said leaflet is responsive to fluid pressure applied against an upstream side of said passageway, and
(b) said leaflets cooperate so that, responsive to changes in said applied fluid pressure, each pivots between a first position where said passageway is substantially fully closed to a second position where said passageway is substantially fully open, thereby defining respective valve closed and valve open configurations relative to said passageway.

9. A method for fabricating a prosthetic heart valve assembly of claim 1 comprising the steps of:
(a) associating each of said leaflets with each of said bearing blocks with the respective ear like projection of each said leaflet associated with a different said bearing recess of each said bearing block so that each said leaflet is pivotable relative to each said bearing block;
(b) associating each of said bearing blocks as so associated with said leaflets with a mold for molding said annular structure so that each said bearing block is located in a different one of said opposed windows of said annular structure as molded in said mold and so that said mold and said bearing blocks in combination define a molding cavity that defines said annular structure; and
(c) molding said annular structure in said molding cavity with said plastic.
